(19) United States
(12) Patent Application Publication
(10) Pub. No.: US 2015/0018890 A1
(43) Pub. Date: Jan. 15, 2015

(54) SCREW IMPLANT WITH REPLACEMENT HEAD FOR HEIGHT ADJUSTMENT AND SIMPLIFIED STORAGE

(71) Applicant: Biomed Establishment, Vaduz (LI)

(72) Inventor: Stefan Ihde, Vrba/Tudorovici (ME)

(73) Assignee: Biomed Establishment, Vaduz (LI)

(21) Appl. No.: 14/258,170

(22) Filed: Apr. 22, 2014

(30) Foreign Application Priority Data
Jul. 12, 2013 (DE) 20 2013 006 276.2

(51) Int. Cl. A61B 17/86

(52) U.S. Cl. CPC A61B 17/8605; A61B 17/8685
USPC 606/306

(57) ABSTRACT

A screw implant a shaft and a threaded area for anchoring in the bone, a head having engagement surfaces or openings for the application of an insertion tool, wherein the shaft and the head are formed in one piece, wherein there is associated with the shaft between the thread and the head at least one separation area where the head with an upper part of the shaft is separated, and a replacement head, which can be fixed to the remaining lower part of the shaft. The replacement head may have an inner drill hole, which is from the size designated to fit a cross section of the shaft and into which the shaft is inserted.

### CROSS REFERENCE

This application claims priority to German Patent Application No. 20 2013 006 276.2, filed Jul. 12, 2013.

### TECHNICAL FIELD OF THE INVENTION

The invention relates to a screw implant, in particular a screw-type implant according to the preamble of claim 1.

### BACKGROUND OF THE INVENTION

In dental implantology screw implants have long been used. Essentially one-piece implants or two-piece implants are used. According to the prior art, these implants are provided in different diameters and heights endosteal and stored. This makes it necessary to keep in stock a significant number of implants in the practitioners office or to plan the needs implants for each patient case, and then selectively implants to order in time for the operation. If errors occur, the correct implant is missing, which is then connected to inconvenience.

Another problem is a priori not predictable decline of bone and mucosa over the years (around the implants), which can lead to unsightly visible metal sites in the mouth.

### SUMMARY OF THE INVENTION

The present invention has the objective to develop implants that are easier to stockpile, and yet universally applicable for different heights, and at the same time solve the problem of bone decline and unwanted visible metal sites.

Basal one-piece screw implants have been developed that find their primary anchorage in the second cortex of the jaw bone. These devices do not need anchorage in the first cortical alveolar ridge. These implants are delivered by the prior art, in different lengths. All screw implants have in common is that they must have areas that allow the application of rotational forces that allow a screw in the implant into the jawbone. Commonly used configurations are for example external squares, hexes or internal hexagons, octagons or internal and external torx-systems. In one-piece implants vertical grooves and external 4-edge has always had to apply.

Since head and implant body are firmly connected, the implant dentists with a broad spectrum of implant lengths are available, for example, 8-50 mm.

In order to reduce this range an implant system was developed in the present case, in addition to the one-piece implant is a replacement to the head (8) is supplied. After insertion of the screw implant in the jaw bone of the shaft with the head attached thereto may be reduced to the required level by the Implantologist separates the stem at the desired location. It remains one anchored part of the implant in the jaw, and stump which projects as per demand out of the jaw.

The replacement head (8) includes an internal drilled hole (11, 11a) which is substantially congruent to the outer shape and the outer diameter of the shaft (5) of the implant (1). The replacement head (11, 11a) is placed on the cut-off piece of the stub shaft (5) and secured, for example with cement or plastics. The diameter of the internal bore (11, 11a) is chosen such that a very thin , precise gap between the cement replacement head (8) and the shaft (5) remains.

The use of a replacement head (5) with smaller, larger or of identical dimensions as the head for inserting, surprisingly is possible because after the insertion of the implant, the one-piece assembled head (3) with its engaging surfaces no longer required, we, and are therefore separated and by replacing the head (8) can be replaced. According to the invention it is not necessary that the shaft has a head (3). It is sufficient if the shaft has on surfaces or elements, which allow screwing of the stem into the jaw bone. Such a shaft without a head can be cut to length according to the invention after being screwed to the required length, prior to the replacement head is mounted on the end of the shaft.

The overlap area between the implant stem (5) and spare head (8) is advantageously at least 3 mm, for which reason and internal drill hole of at least this length (11a, 11) must be present in the replacement head (8).

Through a hole in the head (13) or a cavity superfluous cement can flow out. Thus it is avoided that the cement or adhesive is pressed into the implant mucosa area or even below the mucosa.

If an additional anti-rotation between the implant shaft (5) and the replacement head (8) is desired, shaft and head replacement may have in the overlap region configurations that are not round. For example, an oval-shaped hole 11a is shown in FIG. 4, to which in this case an oval shaft (5) is applied suitably. However, it may also be geometries with several flat sides of the shaft (5) and the internal of the drill hole (11, 11) of the replacement head 8 can be used.

These aspects are merely illustrative of the innumerable aspects associated with the present invention and should not be deemed as limiting in any manner. These and other aspects, features and advantages of the present invention will become apparent from the following detailed description when taken in conjunction with the referenced drawings.

### BRIEF DESCRIPTION OF THE DRAWINGS

Reference is now made more particularly to the drawings, which illustrate the best presently known mode of carrying out the invention and wherein similar reference characters indicate the same parts throughout the views.

FIG. 1 is a view of a basal helical implant according to an embodiment of the present invention.

FIG. 2 is a view of the basal helical implant of FIG. 1 after insertion of the implant.

FIG. 3 is a bottom view of a replacement head for use with a screw implant according to an embodiment.

FIG. 4 is a bottom view of another replacement head for use with a screw implant according to another embodiment.

FIG. 5 is a view of yet another embodiment of a replacement head.

### DETAILED DESCRIPTION

In the following detailed description numerous specific details are set forth in order to provide a thorough understanding of the invention. However, it will be understood by those skilled in the art that the present invention may be practiced without these specific details. For example, the invention is not limited in scope to the particular type of industry application depicted in the figures. In other instances, well-known methods, procedures, and components have not been described in detail so as not to obscure the present invention.

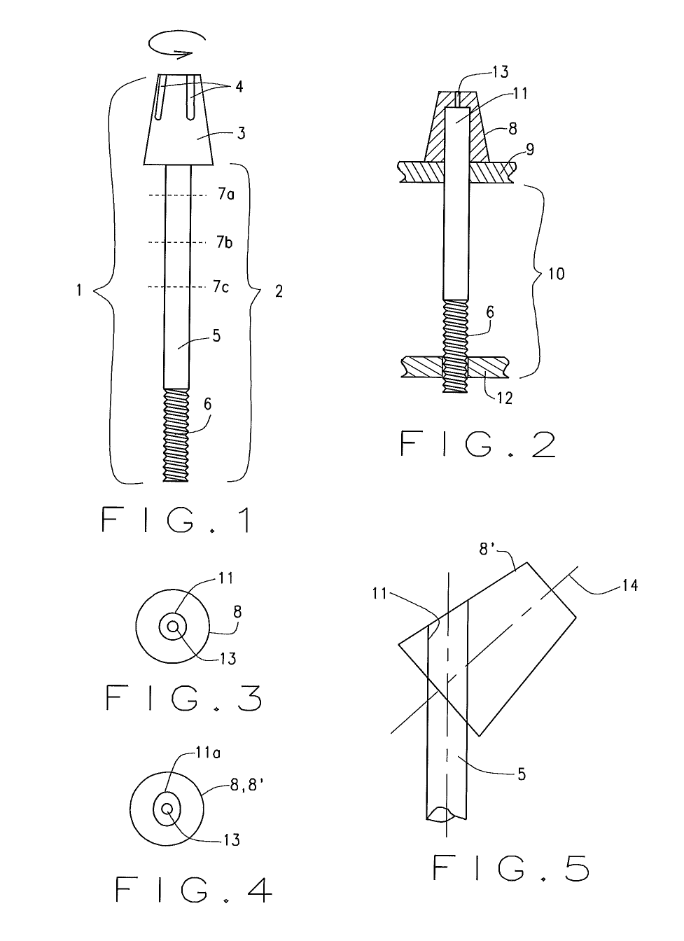

FIG. 1 shows a basal helical implant (1) with endosteal threaded portion (6), a vertical portion in the form of a shank (5) and head (3), wherein the head comprises three engaging grooves (4) or other means, into which a setting tool engages congruently to screw the implant into the jaw bone. The longest possible endosseous implant portion is denoted by (2). After screwing the implant 1 in, the head (3) and the overprojection part of the shaft (5) can be cut off by the surgeon. The positions 7a-c indicate examples of possible separation points, depending on how far the shaft was screwed into the jawbone. The cut off head (3) of the implant 1 is now replaced by the replacement head (8) and it is fixed to the remaining end of the shaft stump (5).

FIG. 2 shows an examples of the invented basal helical implant, which was then cut off after insertion of the implant at the level of the separation point 7a, FIG. 1, that the new, replacement head (8) terminates at the height of the oral mucosa (9). The replacement head (8) has an internal drilling (11), which preferably smaller upwardly (13), thereby a discharge duct for the excess cement is formed. With (10) the endosseous implant portion is designated, as it was available for this implant, the thread (6) is positioned in the second cortical (12). The endosseous implant portion (10) may in other cases shorter or longer, an then the shaft (5) has to be cut off in different separation lines (7a-c). The cement gap between the shaft (5) and the drill hole (11) of the replacement head is for example at most 0.25 mm.

FIG. 3 shows the replacement head 8 from below. Visible is the outflow channel 13 and the internal drill hole (11).

FIG. 4 shows a different embodiment of the replacement head (8) again from below. Visible are the discharge duct 13 and the inner bore 11a, which is here oval.

FIG. 5 shows a particular configuration of the replacement head 8′. In this case, the drill hole (11) does not follow the longitudinal direction of the replacement head (8), but rather is at an angle to the central axis of the replacement head (8), which may improve the usability of the prosthetic implant system, the aesthetic result in many cases.

In summary, the method of use of the invented implant includes the following steps: The threaded portion (6) and the vertical component in the form of the shaft (5) are anchored to the required depth in the jaw bone. For this purpose, the integral head of the implant 3 comprises four engaging surfaces, or other means, into which an insertion tool engages congruently in order to screw the implant into the jaw bone can. After screwing the implant 1 in the head (3) can be cut of the vertical shaft (5) by the surgeon at a required separation point (7a-c) leaving a projecting par of the shaft connected to the implant, depending on how far the shaft was screwed into the jawbone. The separated head (3) of the implant 1 is now replaced by the replacement head 8 which is mounted on the remaining end of the shaft 5. The replacement head 8 is placed and fixed on the end of the shaft 5. In a further advantageous embodiment of the replacement head 8′ is formed so that there is an angle between the axial direction of the shaft and the axial direction of the replacement head. This angle causes angulation of the replacement head 8′ with respect to the axial direction of the implant 1.

The preferred embodiments of the invention have been described above to explain the principles of the invention and its practical application to thereby enable others skilled in the art to utilize the invention in the best mode known to the inventors. However, as various modifications could be made in the constructions and methods herein described and illustrated without departing from the scope of the invention, it is intended that all matter contained in the foregoing description or shown in the accompanying drawings shall be interpreted as illustrative rather than limiting. Thus, the breadth and scope of the present invention should not be limited by the above-described exemplary embodiment, but should be defined only in accordance with the following claims appended hereto and their equivalents.

### LIST OF REFERENCE NUMERALS

1 One-piece screw implant basal
2 Endosteal portion of the screw implant
3 implant head
4 engaging grooves in the implant head
5 shaft of the implant
6 threads of the implant for anchoring in the second cortex
7a-7b possible separation points
8, 8′ Replacement Head
9 mucosa (range)
10 Endosteal portion of the implant
11, 11a internal drill hole in the replacement head
12 2nd cortical (opposite cortical bone) of the jaw bone
13 spillway, duct
14 central axis

1. A screw implant comprising:
a shaft and a threaded area for anchoring in the bone,
a head having engagement surfaces or openings for the application of an insertion tool,
wherein the shaft and the head are formed in one piece,
wherein there is associated with the shaft between the thread and the head at least one separation area where the head with an upper part of the shaft is separated, and
a replacement head, which can be fixed to the remaining lower part of the shaft.

2. The screw implant according to claim 1, wherein the replacement has an inner drill hole, which is from the size designated to fit a cross section of the shaft and into which the shaft is inserted.

3. The screw implant according to claim 2, wherein the replacement and the shaft have an overlapping region in the region of the inner drill hole of at least 3 mm.

4. The screw implant according to claim 1, wherein the replacement is affixed on the shaft by cement or plastics.

5. The screw implant according to claim 2, wherein the replacement is provided with a further drill hole or cavity for the outflow of excess cement in the region of the inner drill hole.

6. The screw implant according to claim 2, wherein a cross section of the inner drill hole in the replacement head is formed non-circular.

7. The screw implant according to claim 2, wherein the inner drill hole is arranged at an angle to a central axis of the replacement head.

8. A replacement for a screw implant having a shaft, comprising:
an inner drill hole for mounting on the shaft of the screw implant.

9. A replacement head for a screw implant having a shaft, comprising:
an inner drill hole for mounting on the shaft of the screw implant, and
wherein the inner drill hole is arranged at an angle to the central axis of the replacement head.

10. The replacement head according to claim 8, wherein the replacement head and the shaft have an overlapping region in the region of the inner drill hole of at least 3 mm.

11. The replacement head according to claim 8, wherein the replacement head is affixed on the shaft by cement or plastics.

12. The replacement head according to claim 8, wherein the replacement head is provided with a further drill hole or cavity for the outflow of excess cement in the region of the inner drill hole.

13. The replacement head according to claim 8, wherein a cross section of the inner drill hole in the replacement head is formed non-circular.

14. The screw implant according to claim 2, wherein the inner drill hole is arranged at an angle to a central axis of the replacement head.

15. The replacement head according to claim 9, wherein the replacement head and the shaft have an overlapping region in the region of the inner drill hole of at least 3 mm.

16. The replacement head according to claim 9, wherein the replacement head is affixed on the shaft by cement or plastics.

17. The replacement head according to claim 9, wherein the replacement head is provided with a further drill hole or cavity for the outflow of excess cement in the region of the inner drill hole.

18. The replacement head according to claim 9, wherein a cross section of the inner drill hole in the replacement head is formed non-circular.
